(19) United States
(12) Patent Application Publication
(10) Pub. No.: US 2004/0052844 A1
(43) Pub. Date: Mar. 18, 2004

(54) Time-controlled, sustained release, pharmaceutical composition containing water-soluble resins

(76) Inventors: Fang-Hsiung Hsiao, Tainan Hsien (TW); Sung-Jen Chen, Hsin-Ying City (TW); Chien-Chu Lin, Hsin-Ying City (TW); Ya-Ching Changchien, Kaohsiung (TW)

Correspondence Address: Rabin & Berdo, P.C., Suite 500, 1101 14th Street, Washington, DC, 20005, US

(21) Appl. No.: 10/243,932

(22) Filed: Sep. 16, 2002

(51) Int. Cl. A61K 9/24; A61K 9/14

(52) U.S. Cl. 424/471

(57) ABSTRACT

The invention relates to a time-controlled, sustained release, pharmaceutical composition containing water-soluble resins, comprising:

(a) a core, comprising 1˜10% by weight of active ingredients, 0˜80% by weight of diluents, 1˜50% by weight of water-soluble resins, 5˜30% by weight of plasticizers, 0˜50% by weight of adhesives and 0.5˜5% by weight of lubricants, based on the total weigh of the pharmaceutical composition; and

(b) a film coating, comprising 0.5˜80% by weight of film coating forming agents, 0.5˜30% by weight of plasticizers and 0.5˜20% by weight of anti-sticking agents or other diluents, based on the total weight of the pharmaceutical composition.

### BACKGROUND OF THE INVENTION

1. Field of the Invention

The present invention relates to a time-controlled, sustained release, pharmaceutical composition containing water-soluble resins. Specifically, the present invention relates to a time-controlled, sustained release, pharmaceutical composition on containing water-soluble resins, which provides a special time to release and a constant level of a drug in a subject.

2. Description of Related Art

Due to the fact that many pharmaceutically active ingredients have a short biological half-life those pharmaceutically active ingredients need to be administered several times a day in clinic applications to achieve the desired of treatment effect. Sustained release pharmaceutical compositions that provide a constant level of drugs in a subject over an extended period of time have been developed in the art to overcome the foregoing issue. The relevant documents concerning sustained release pharmaceutical compositions are cited below.

Taiwan Patent Publication No. 393320 relates to an oral hydrogel control release pharmaceutical tablet comprising: (1) at least one medicament; (2) additives for infiltrating water into the tablets; and (3) high molecular substances for forming hydrogel. Said pharmaceutical tablet has the ability to gelatinize completely during staying in the upper alimentary tract, and to release the medicine in the colon of the lower alimentary tract. The main site for absorbing the oral hydrogel control release pharmaceutical tablet is the lower alimentary tract.

U.S. Pat. No. 5.024,843 discloses an oral hypoglycemic glipizide granule. U.S. Pat. No. 5,091,190 describes a delivery system for administration of a blood-glucose lowering drug. Furthermore, a dosage form for administering oral hypoglycemic glipizide and a method for controlling hyperglycemia are disclosed in U.S. Pat. No. 5,545,413. Those patents relate to providing a dosage form to release glipizide at a controlled rate for blood-glucose lowering therapy.

U.S. Pat. No. 5,273,758 discloses a direct compression process for preparing a tableted pharmaceutical dosage form, consisting of the steps: a) blending a crystallized therapeutic medicament with a directly compression vehicle consisting essentially of polyethylene oxide, in the absence of solvent or heat, to form a composition in which the medicament is dispersed; and b) compressing the resultant composition under a sufficient pressure to form a tablet. According to the disclosure of this patent, the releasing rate of a drug is controlled by using polyethylene oxide.

U.S. Pat. No. 5,593,694 relates to a zero-order dissolution, sustained release tablet comprising a base tablet comprising a water-swellable gelling agent and a pharmaceutically active ingredient dispersed homogeneously in said gelling agent, said base tablet being coated with a film coating composition prepared by dissolving one or two members selected from the group consisting of ethylcellulose and acetylcellulose in an organic solvent. This patent does not use polyethylene oxide.

U.S. Pat. No. 5,783,212 describes a controlled release pharmaceutical tablet comprising a first barrier layer comprising a first swellable, erodible polymer; a drug layer comprising a second swellable, erodible polymer; and a second barrier layer comprising a third swellable, erodible polymer, wherein said first and second barrier layers are adapted to swell and to erode faster than said drug layer; and said faster swellability and erodibility of said first and second barrier layers adapted to increase drug delivery from the onset of dissolution. The average molecular weight of polyethylene oxide used in this patent is between 1×10⁶ and 2×10⁶.

U.S. Pat. No. 6,056,977 discloses a sustained release oral solid dosage form, wherein the dosage form contains a therapeutically effective amount of a sulfonylurea or a salt or derivative thereof in the matrix. Further, the use of an aqueous alkalizing medium affords substantially complete bioavailability of the drug from the matrix of the tablet. The tablet cores may optionally be coated with a coating material in tile range of 2% to 10% with an enteric material or with a water-insoluble material like ethyl cellulose. This patent does not use polyethylene oxide.

U.S. Pat. No. 6,090,411 relates to a swellable hydrophollic matrix tablet that delivers drugs in a controlled manner over a long period of time. More specifically, the drug is dispersed in a matrix composed of HPMC or polyethylene oxide, in the presence of a salt or a combination of salts.

WO 96/26718 describes a controlled release tablet including a pharmaceutical agent and an excipient. The excipient includes at least about 50% of a water-swellable polymer and a lubricant. The average molecular weight of polyethylene oxide used in this patent is between 900,000 and 4,000,000.

WO 97/18814 discloses a controlled release pharmaceutical formulation for oral administration consisting essentially of: an active drug compound; low molecular weigh polyethylene oxide; hydroxypropylmethyl cellulose; tabletting excipients; and optionally one or more enteric polymers. The number average molecular weight of polyethylene oxide used in this patent is between 20,000 and 500,000.

All references as cited herein are incorporated by reference.

A time-controlled, sustained release, pharmaceutical composition containing water-soluble resins can deliver a drug such as glipizide at a controlled rate to a subject. Glipizide is an oral blood glucose lowering drug and is a kind of sulfonylureas. Glipizide is useful for the treatment of non-insulin-dependent diabetes or maturity-onset diabetes. Glipizide can stimulate insulin secretion from the pancreas to reduce the level of blood glucose. The functions of glipizide outside the pancreas include increasing the sensitivity to the production of insulin and reducing the production of glycogen. Furthermore, glipizide has an important influence on the effect of lowering blood glucose.

Sustained release tablets comprising sulfonylureas are commercially available from Pfizer Inc., for example, Glucotrol XL Tab. (Glipizide 2.5 mg, 5 mg and 10 mg). Those tablets are OROS controlled release dosage forms. The core of said tablets consists of a layer containing a drug and a layer without a drug and is coated with a semi-permeable membrane that is drilled by using a laser. Tile release mechanism of such a tablet releases the drug through the pores of the semi-permeable membrane due to the difference in the osmotic pressure between the inside and outside of the tablet and the swelling of the layer without a drug to achieve a zero-order release effect for the drug. However, the process for preparing such OROS controlled release dosage forms is complicated. Furthermore, drilling semi-permeable membranes requires the use of a laser or other mechanical means. It is difficult to ensure that the location of each pore in each tablet is the same (i.e. on the side of the layer containing a drug) and each tablet has a uniform pore size and depth. Therefore, it is not possible to assure a consistent dissolution profile for each tablet. However, the process for preparing the time-controlled, sustained release, pharmaceutical composition in accordance with the present invention comprises mixing water-soluble resins with appropriate excipients and then directly tabletting to form a tablet. Alternatively, appropriate binder solutions for granulation can be added to the mixture, and after drying, the lubricated granules are tabletted. The result of an in vitro dissolution test for the naked tablet shows a substantially zero-order dissolution profile. The naked tablet is coated with one or more film coatings prepared by admixing water-soluble polymers and water-insoluble polymers. The time-controlled, sustained release, pharmaceutical composition in accordance with the present invention may have a different drug releasing time depending on the ratios of the components in the pharmaceutical composition. The main advantage of the present invention is that the process for preparing the time-controlled, sustained release, pharmaceutical composition is simple and no special equipment is required.

The novel time-controlled, sustained release, pharmaceutical composition in accordance with the present invention provides a constant level of a drug in a subject. Furthermore, the time-controlled, sustained release, pharmaceutical composition exhibits a good capability of delivering certain drugs that need to be absorbed at specific sites of a body or releasing active drugs over an delayed period of time.

### SUMMARY OF THE INVENTION

An object of the present invention is to provide a time-controlled, sustained release, pharmaceutical composition comprising water-soluble resins, which provides a constant releasing rate of a drug in the gastrointestinal tract for an extended time.

Another object of the present invention is to provide a time-controlled, sustained release, pharmaceutical composition comprising sulfonylureas as the active ingredients, which is suitable for once-a-day administration and has bioavailability.

Another object of the present invention is to provide a time-controlled, sustained release, pharmaceutical composition comprising glipizide as an active ingredient, which is suitable for once-a-day administration and has bioavailability.

Other objectives, advantages and novel features of the present invention will become more apparent from the following detailed description when taken in conjunction with the accompanying drawings.

### BRIEF DESCRIPTION OF THE DRAWINGS

FIG. 1 is a graph showing the dissolution rates of the time-controlled, sustained release, pharmaceutical compositions obtained from Examples 3, 4 and 5 of the present invention and the commercial pharmaceutical composition (Glucotrol XL Tab., Glipizide 5 mg, Pfrizer Inc.) in a buffer solution (pH7.5).

FIG. 2 is a graph showing the plasma concentrations versus time for the time-controlled, sustained release, pharmaceutical composition obtained from Example 5 of the present invention and the commercial pharmaceutical composition (Glucotrol XL Tab., Glipizide 5 mg, Pfrizer Inc.).

FIG. 3 is a graph showing the fractions released versus time for Diltiazem HCl from tablets coated with various amounts of Aquacoat® ECD-30.

### DETAILED DESCRIPTION OF THE INVENTION

The term “sustained release” as used herein means the release of an active ingredient at a rate such that levels of the active ingredient in the blood are maintained within a therapeutic range but below toxic levels over an extended period of time such as 12 to 24 hours or longer.

The term “time-controlled, sustained release” as used herein means that the sustained release of a drug in vivo can be controlled by use of certain techniques.

The term “bioavailability” as used herein means the physiological availability of an amount of a given drug. That is, the drug is released from the sustained release dosage form and becomes available at the action site of the drug.

The present invention relates to a time-controlled, sustained release, pharmaceutical composition containing water-soluble resins, which provides a constant releasing rate of a drug over an extended period of time.

The time-controlled, sustained release, pharmaceutical composition consists of a core and a film coating. The core can be a tablet, a minitablet or a pellet. The core contains 1˜10% by weight of active ingredients, 0˜80% by weight of diluents, 1˜50% by weight of water-soluble resins, 5˜30% by weight of plasticizers, 0˜50% by weight of adhesives and 0.5˜5% by weight of lubricants, based on the total weight of the pharmaceutical composition. The film coating contains 0.5˜80% by weight of film coating forming agents, 0.5˜30% by weight of plasticizers and 0.5˜20% by weight of anti-sticking agents or other diluents, based on the total weight of the pharmaceutical composition.

In a preferred embodiment of the present invention, the active ingredients are selected from the group consisting of sulfonylurea drugs, dipyridamole, dichlofenac sodium and diltiazem hydrochloride. A preferred example of the sulfonylurea drug is glipizide.

The process for preparing the core of the pharmaceutical composition in accordance with the present invention can be dry granulation, wet granulation, fluidized-bed granulation or compression granulation. After adding lubricants to the granules, the granules can be compressed into tablets or pelletized into pellets. Water-soluble resins, plasticizers, adhesives, water, appropriate organic solvents or the mixtures thereof and/or the emulsified or non-emulsified suspension of the mixtures can be added to the solution used for granulation.

The core of the pharmaceutical composition can be coated with one or more layers of film coatings. The process for preparing the film coating in accordance with the present invention can be known coating methods, such as a pan coating method or fluidized-bed coating method. Appropriate organic solvents or the mixtures of organic solvents and/or an emulsified or non-emulsified suspension of polymers are applied to the surface of the core to form a core coated with one or more layers of film coatings.

The diluents used in the core of the pharmaceutical composition may be chosen from those known in the pharmaceutical art. In a preferred embodiment, the diluents are selected from the group consisting of saccharides, starches, sodium cellulose, alkaline metal salts and alkaline earth metal salts.

The water-soluble resins used in the core of the pharmaceutical composition may be chosen from those known in the pharmaceutical art. In a preferred embodiment, the water-soluble resin is polyethylene oxide. The molecular weight of the polyethylene oxide is preferably between 100,000 and 7,000,000, more preferably, between 300,000 and 1,000,000, and most preferably, is 600,000.

The adhesives used in the core of the pharmaceutical composition may be chosen from those known in the pharmaceutical art. In a preferred embodiment, the adhesives are selected from the group consisting of polyvinyl pyrrolidone (PVP), gelatin, hydroxyethyl cellulose (HEC), hydroxypropyl cellulose (HPC), hydroxypropyl methyl cellulose (HPMC), vinyl acetate (VA), polyvinyl alcohol (PVA), methyl cellulose (MC), ethyl cellulose (EC), hydroxypropyl methyl cellulose phthalate (HPMCP), cellulose acetate phthalates (CAP), xanthan gum, alginic acid, salts of alginic acid, the copolymer of methyl acrylic acid/methyl methacrylate (Eudragit®), polyvinyl acetate phthalate (PVAP) and polyvinyl acetate (PVAc).

The lubricants used in the core of the pharmaceutical composition may be chosen from those known in the pharmaceutical art. In a preferred embodiment, the lubricants are selected from the group consisting of talc, stearic acid, stearate, sodium stearyl fumarate, glyceryl behenate, kaolin and aerosil.

The film coating forming agents used in the film coating of the pharmaceutical composition may be chosen from those known in the pharmaceutical art. In a preferred embodiment, the film coating forming agents are selected form the group consisting of the copolymer of methyl acrylic acid/methyl methacrylate such as Eudragit® RL/RS (commercially available from Rohm Pharma.), Aquacoat® or Kollicoat®, polyvinyl pyrrolidone (PVP), gelatin, hydroxyethyl cellulose (HEC), hydroxypropyl cellulose (HPC), hydroxypropyl methyl cellulose (HPMC), vinyl acetate (VA), polyvinyl alcohol (PVA), methyl cellulose (MC), ethyl cellulose (EC), hydroxypropyl methyl cellulose phthalate (HPMCP), cellulose acetate phthalates (CAP), alginic acid, salts of alginic acid, polyvinyl acetate phthalate (PVAP) and polyvinyl acetate (PVAc).

The plasticizers used in the present invention may be chosen from those known in the pharmaceutical art. In a preferred embodiment, the plasticizers are selected from the group consisting of glycerin, polyethylene glycol, triethyl citrate, tributyl citrate, propyl triacetate and castor oil.

The other diluents used in the film coating of the pharmaceutical composition may be chosen from those known in the pharmaceutical art. In a preferred embodiment, the other diluents are selected from the group consisting of lactose, starch, mannitol, sodium carboxymethyl cellulose, sodium starch, milcrocrystalline cellulose and pigments.

The anti-sticking agents used in the film coating of the pharmaceutical composition may be chosen from those known in the pharmaceutical art. In a preferred embodiment, the anti-sticking agents are selected from the group consisting of talc, stearic acid, stearate, sodium stearyl fumarate and glyceryl behenate.

The solvents used in the present invention may be chosen from those known in the pharmaceutical art. In a preferred embodiment, the solvents are selected from the group consisting of water, alcohol, acetone, isopropanol, dichloromethane and combinations thereof.

The following examples are given to illustrate the characteristics and features of the composition in accordance with the present invention. However, the invention should not be construed to be limited to these examples.

### EXAMPLE 1

Formulation:

(1) Tablet Core:

Preparation:

50 g of dipyridamole, 210 g of lactose, 80 g of polyethylene glycol 6000, 280 g of polyethylene oxide (M.W.=600,000) and 320 g of hydroxypropylmethyl cellulose (M.W.=20,000) were respectively passed through an appropriate mesh of a sieve and then combined in a granulation machine for granulation. After carrying out the granulation, the resultant granules were pelletized in a pelleting machine to form pellet cores.

(2) Film Coating:

| Components: | | |
|---|---|---|
| Aquacoat ® ECD-30 | 132 | g |
| Triethyl citrate | 24.8 | g |
| Talc | 18 | g |
| Solvent | 200 | ml |

Preparation:

132 g of Aquacoat® ECD-30, 24.8 g of triethyl citrate and 18 g of talc were dissolved in 200 ml of a solvent. Subsequently, the aqueous solution was sprayed onto the surface of the pellet cores.

### EXAMPLE 2

Formulation:

(1) Tablet Core:

Preparation:

100 g of dichlofenac sodium, 200 g of lactose, 80 g of polyethylene glycol 6000, 240 g of polyethylene oxide (M.W.=600,000) and 310 g of hidroxypropylmethyl cellulose (M.W.=20,000) were respectively passed through an appropriate mesh of a sieve, and then combined and homogeneously mixed in a mixer. Subsequently, 18 g of magnesium stearate was added to the mixture and then the mixture was homogeneously blended. The resultant mixture was compressed into tablets in a tabletting machine. Each tablet had a weight of 190 mg and a diameter of 8 mm.

(2) Film Coating:

| Components: | | |
|---|---|---|
| Eudragit ® RS30D | 140 | g |
| Hydroxypropylmethyl cellulose | 12 | g |
| Triethyl citrate | 8.4 | g |
| Talc | 16 | g |
| Solvent | 250 | ml |

Preparation:

12 g of hydroxypropylmethyl cellulose was dissolved in 250 ml of a solvent. 140 g of Eudragit® RS30D, 8.4 g of triethyl citrate and 16 g of talc were then dissolved in the aqueous solution. Subsequently, the aqueous solution was sprayed onto the surface of the tablets.

### EXAMPLE 3

Formulation:

(1) Tablet Core:

Preparation:

25 g of glipizide, 223 g of lactose, 90 g of polyethylene glycol 6000, 270 g of polyethylene oxide (M.W.=600,000) and 315 g of hydroxypropylmethyl cellulose (M.W.=20,000) were respectively passed through an appropriate mesh of a sieve and then homogeneously mixed in a mixer. Subsequently, 18 g of magnesium stearate was added to the mixture and then the mixture was homogeneously blended. The resultant mixture was compressed into tablets in a tabletting machine. Each tablet had a weight of 190 mg and a diameter of 8 mm.

(2) Film Coating:

| Components: | | |
|---|---|---|
| Aquacoat ® ECD-30 | 140 | g |
| Hydroxypropylmethyl cellulose | 32 | g |
| Triethyl citrate | 16 | g |
| Talc | 16 | g |
| Solvent | 200 | ml |

Preparation:

32 g of hydroxypropylmethyl cellulose was dissolved in 200 ml of a solvent. 140 g of Aquacoat® ECD-30, 16 g of triethyl citrate and 16 g of talc were then dissolved in the aqueous solution. Subsequently, the aqueous solution as sprayed onto the surface of the tablets.

### EXAMPLE 4

Formulation:

(1) Tablet Core:

Preparation:

25 g of glipizide, 223 g of lactose, 90 g of polyethylene glycol 6000, 270 g of polyethylene oxide (M.W.=600,000) and 315 g of hydroxypropylmethyl cellulose (M.W.=20,000) were respectively passed through an appropriate mesh of a sieve and then homogeneously mixed in a mixer. Subsequently, 18 g of magnesium stearate was added to the mixture and then the mixture was homogeneously blended. The resultant mixture was compressed into tablets in a tabletting machine. Each tablet has a weight of 190 mg and a diameter of 8 mm.

(2) Film Coating:

| Components: | | |
|---|---|---|
| Kollicoat ® SR 30D | 132 | g |
| Triethyl citrate | 24.8 | g |
| Talc | 18 | g |
| Solvent | 200 | ml |

Preparation:

132 g of Kollicoat® SR 30D, 24.8 g of triethyl citrate and 18 g of talc were dissolved in 200 ml of solvent. Subsequently, the aqueous solution was sprayed on the surface of the tablets.

### EXAMPLE 5

Formulation:

(1) Tablet Core:

Preparation:

25 g of glipizide, 223 g of lactose, 90 g of polyethylene glycol 6000, 270 g of polyethylene oxide (M.W.=600,000) and 315 g of hydroxypropylmethyl cellulose (M.W.=20,000) were respectively passed through an appropriate mesh of a sieve and then homogeneously mixed in a mixer. Subsequently, 18 g of magnesium stearate was added to the mixture and then the mixture was homogeneously blended. The resultant mixture was compressed into tablets in a tabletting machine. Each tablet had a weight of 190 mg and a diameter of 8 mm.

(2) Film Coating:

| Components: | | |
|---|---|---|
| Eudragit ® RS 30D | 140 | g |
| Hydroxypropylmethyl cellulose | 12 | g |
| Triethyl citrate | 8.4 | g |
| Talc | 16 | g |
| Solvent | 250 | ml |

Preparation:

12 g of hydroxypropylmethyl cellulose was dissolved in 250 ml of a solvent. 140 g of Eudragit® RS30D, 8.4 g of triethyl citrate and 16 g of talc were then dissolved in the aqueous solution. Subsequently, the aqueous solution was sprayed onto the surface of the tablets.

### EXAMPLE 6

Dissolution Test:

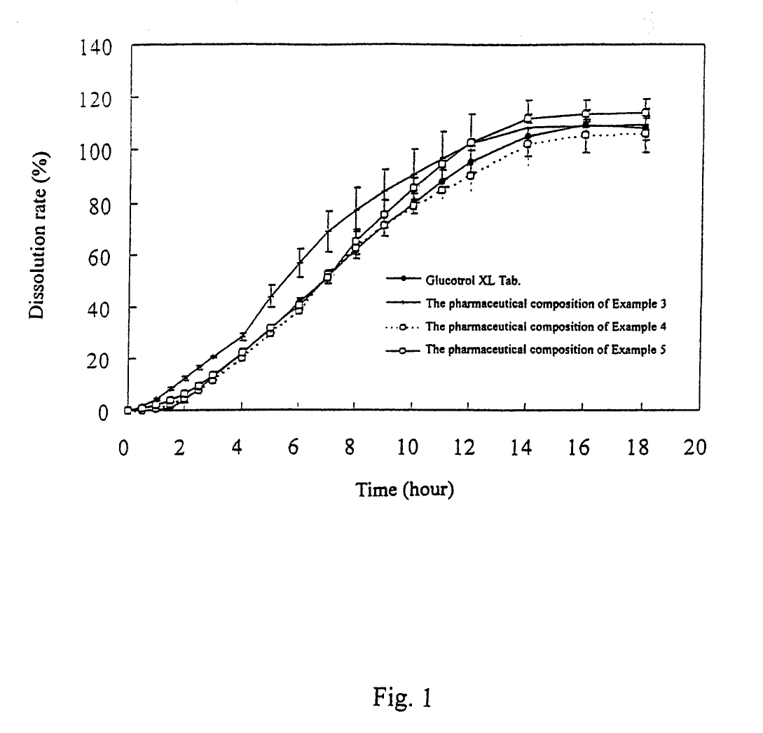

The time-controlled, sustained release, pharmaceutical compositions obtained from Examples 3, 4 and 5 and the commercial pharmaceutical composition (Glucotrol XL Tab., Glipizide 5 mg, Pfrizer Inc.) were tested. The dissolution test was carried out using pH 7.5 buffer solution. The dissolution rate was measured and the results are presented graphically in FIG. 1.

### EXAMPLE 7

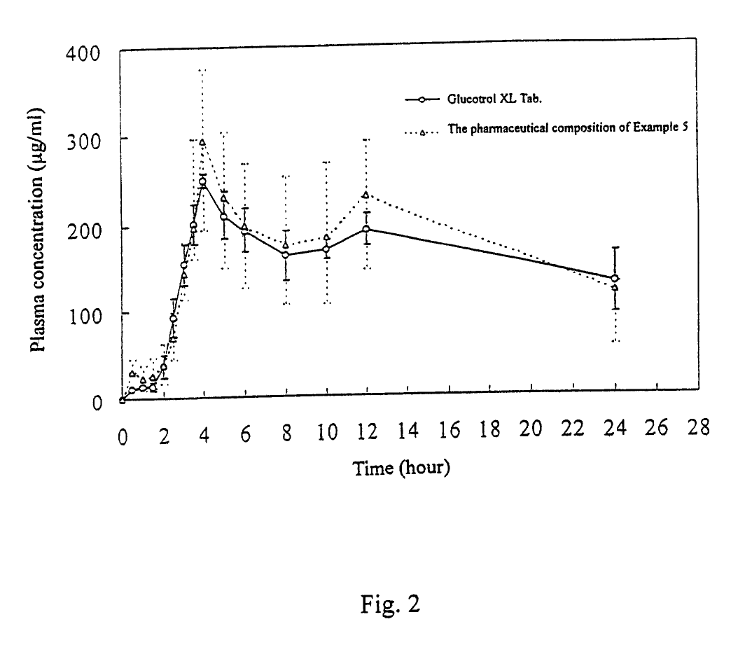

Four healthy adult subjects were divided into two groups, and 10 mg glipizide dosage of the time-controlled, sustained release, pharmaceutical composition obtained from Example 5 and 10 mg glipizide dosage from the commercial pharmaceutical composition (Glucotrol XL Tab., Glipizide 5 mg, Pfrizer Inc.) were respectively administered to the subjects in each group. Within 24 hours after administration, the blood samples of the subjects were collected at predetermined times. The plasma concentration of glipizide in each blood sample was measured. The results are listed in Table 1 and presented graphically in FIG. 2.

TABLE 1
Plasma concentrations of the
time-controlled, sustained release,
pharmaceutical composition obtained
from Example 5 of the present invention
and the commercial pharmaceutical composition
(Glucotrol XL Tab., Glipizide 5 mg, Pfrizer Inc.)(n = 4)

| | The pharmaceutical |
|---|---|
| Glucotrol XL Tab. | composition of Example 5 |

| | Average | | Average | |
|---|---|---|---|---|
| Time | Concentration | | Concentration | |
| (hr) | (μg/ml) | S.D. | (μg/ml) | S.D. |
| 0 | 0.00 | 0.00 | 0.00 | 0.00 |
| 0.5 | 11.15 | 0.00 | 31.06 | 0.00 |
| 1 | 13.00 | 0.99 | 23.97 | 14.38 |
| 1.5 | 13.43 | 4.63 | 26.19 | 14.30 |
| 2 | 36.44 | 13.45 | 38.36 | 21.30 |
| 2.5 | 92.47 | 22.63 | 68.86 | 24.95 |
| 3 | 154.23 | 24.00 | 143.40 | 29.88 |
| 3.5 | 200.92 | 22.70 | 197.59 | 37.05 |
| 4 | 249.56 | 7.50 | 293.99 | 98.41 |
| 5 | 210.09 | 26.43 | 230.69 | 81.10 |
| 6 | 193.10 | 25.44 | 199.22 | 73.10 |
| 8 | 163.02 | 29.54 | 175.69 | 69.03 |
| 10 | 169.48 | 11.95 | 183.87 | 77.70 |
| 12 | 192.34 | 18.41 | 230.53 | 84.51 |
| 24 | 128.68 | 35.83 | 118.63 | 62.44 |

From a comparison of the results in Table 1, the Tₘₐₓ value and Cₘₐₓ value of the time-controlled, sustained release, pharmaceutical composition obtained from Example 5 in vivo is consistent with that of the commercial pharmaceutical composition containing glipizide. Therefore, the sustained release capability of the time-controlled, sustained release, pharmaceutical composition of the present invention is equivalent to the sustained release capability of the commercial pharmaceutical composition containing glipizide.

### EXAMPLE 8

(1) Tablet Core:

Preparation:

30 g of diltiazem HCl, 340 g of lactose, 140 g of polyethylene glycol 6000 and 463 g of hydroxypropylmethyl cellulose (M.W.=20,000) were respectively passed through an appropriate mesh of a sieve and then homogeneously mixed in a mixer. Subsequently, 26 g of magnesium stearate was added to the mixture and the mixture was subsequently homogeneously blended. The resultant mixture was compressed into tablets in a tabletting machine. Each tablet had a weight of 190 mg and a diameter of 8 mm.

(2) Film Coating:

The components of the film coating:

| Components | I | II | III | IV | V |
|---|---|---|---|---|---|
| Aquacoat ® ECD-30 | 166.67 | 250.0 | 333.33 | 416.67 | 250.0 |
| Triethyl citrate | 10 | 15 | 20 | 25 | 7.5 |
| Talc | 40 | 60 | 80 | 100 | 60 |
| Polysorbate 80 | 0.1 | 0.15 | 0.2 | 0.25 | 0.075 |
| Tablet core | 1000 | 1000 | 1000 | 1000 | 1000 |

Preparation:

An appropriate amount of triethyl citrate was dissolved in an appropriate amount of deionized water containing polysorbate 80. An appropriate amount of talc was passed through a 150 mesh sieve and then added to the aqueous solution to form a homogeneous solution. Subsequently, an appropriate amount of Aquacoat® ECD-30 was slowly added to the solution and stirred for 30 minutes. The resultant aqueous solution was sprayed onto the surface of the tablet core.

### EXAMPLE 9

Dissolution Test:

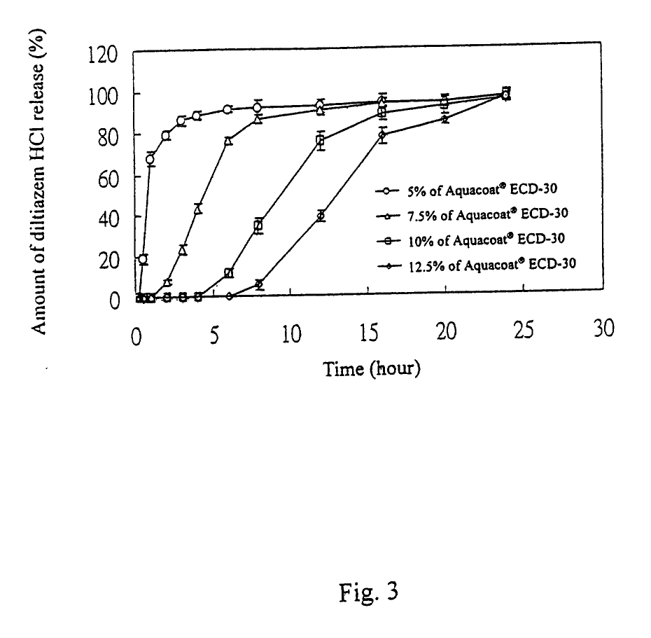

The dissolution test was carried out on the tablets obtained from the procedure in Example 8. The dissolution test was carried out using USP dissolution apparatus (“Paddle Method”, 24^{th} edition) in terms of pH-change method. The first 2 hours in gastric fluid, simulated solution, after 2 hours, changed to intestinal fluid, simulated solution, the dissolution test was carried out for further 24 hours. The dissolution rate was measured. The results are listed in Table 2 and presented graphically in FIG. 3.

TABLE 2
The regression results of the fractions released
versus time for Diltiazem HCl from tablets coated with
various amounts of Aquacoat ® ECD-30.

| Time | Amount of Aquacoat ® ECD-30(%) | | | |
|---|---|---|---|---|
| (hour) | 5% | 7.5% | 10% | 12.5% |
| 0.25 | 0.92 | — | — | — |
| | (0.02)^{a} | | | |
| 0.5 | 18.61 | — | — | — |
| | (2.55) | | | |
| 1 | 68.02 | — | — | — |
| | (3.78) | | | |
| 2 | — | 7.61 | — | — |
| | | (1.13) | | |
| 3 | — | 22.85 | — | — |
| | | (2.19) | | |
| 4 | — | 43.06 | — | — |
| | | (2.26) | | |
| 6 | — | 76.16 | 11.24 | — |
| | | (1.89) | (2.08) | |
| 8 | — | — | 34.1 | 5.37 |
| | | | (3.61) | (2.37) |
| 12 | — | — | 75.66 | 38.31 |
| | | | (4.58) | (2.84) |
| 16 | — | — | — | 77.62 |
| | | | | (3.64) |
| Slope (% hr⁻¹) | 90.8 | 17.33 | 10.63 | 9.03 |
| Intercept (%) | −23.79 | −27.58 | −52.29 | −67.94 |
| r | 0.9971 | 0.9991 | 0.9997 | 0.9987 |
| Lag time^{b} (h) | 0.2620 | 1.5915 | 4.8915 | 7.5238 |

^{a}Standard deviation (n = 6)
^{b}Lag time = −(Intercept/Slope)

According to Table 2, the film coating in accordance with the present invention also has an effect on the sustained release of a drug.

It must be understood that the specification and examples are illustrative but do not limit the scope of the present invention.

What is claimed is:

1. A time-controlled, sustained release, pharmaceutical composition containing water-soluble resins, comprising:
(a) a core, comprising 1˜10% by weight of active ingredients, 0˜80% by weight of diluents, 1˜50% by weight of water-soluble resins, 5˜30% by weight of plasticizers, 0˜50% by weight of adhesives and 0.5˜5% by weight of lubricants, based on the total weight of the pharmaceutical composition; and
(b) a film coating, comprising 0.5˜80% by weight of film coating forming agents, 0.5˜30% by weight of plasticizers and 0.5˜20% by weight of anti-sticking agents or other diluents, based on the total weight of the pharmaceutical composition.

2. The pharmaceutical composition according to claim 1, wherein the active ingredients are selected from the group consisting of sulfonylurea drugs, dipyridamole, dichlofenac sodium and diltiazem hydrochloride.

3. The pharmaceutical composition according to claim 2, wherein the sulfonylurea drug is glipizide.

4. The pharmaceutical composition according to claim 1, wherein the diluents used in the core are selected from the group consisting of saccharides, starches, sodium cellulose, alkaline metal salts and alkaline earth metal salts.

5. The pharmaceutical composition according to claim 1, wherein the water-soluble resin used in the core is polyethylene oxide.

6. The pharmaceutical composition according to claim 5, wherein the polyethylene oxide has a molecular weight between 100,000 and 7,000,000.

7. The pharmaceutical composition according to claim 6, wherein the polyethylene oxide has a molecular weight between 300,000 and 1,000,000.

8. The pharmaceutical composition according to claim 7, wherein the polyethylene oxide has a molecular weight of 600,000.

9. The pharmaceutical composition according to claim 1, wherein the adhesives used in the core are selected form the group consisting of polyvinyl pyrrolidone (PVP), gelatin, hydroxyethyl cellulose (HEC), hydroxypropyl cellulose (HPC), hydroxypropyl methyl cellulose (HPMC), vinyl acetate (VA), polyvinyl alcohol (PVA), methyl cellulose (MC), ethyl cellulose (EC), hydroxypropyl methyl cellulose phthalate (HPMCP), cellulose acetate phthalates (CAP), xanthan gum, alginic acid, salts of alginic acid, the copolymer of methyl acrylic acid/methyl methacrylate (Eudragit®), polyvinyl acetate phthalate (PVAP) and polyvinyl acetate (PVAc).

10. The pharmaceutical composition according to claim 1, wherein the lubricants used in the core are selected from the group consisting of talc, stearic acid, stearate, sodium stearyl fumarate, glyceryl behenate, kaolin and aerosil.

11. The pharmaceutical composition according to claim 1, wherein the film coating forming agents are selected form the group consisting of the copolymer of methyl acrylic acid/methyl methacrylate, polyvinyl pyrrolidone (PVP), gelatin, hydroxyethyl cellulose (HEC), hydroxypropyl cellulose (HPC), hydroxypropyl methyl cellulose (HPMC), vinyl acetate (VA), polyvinyl alcohol (PVA), methyl cellulose (MC), ethyl cellulose (EC), hydroxypropyl methyl cellulose phthalate (HPMCP), cellulose acetate phthalates (CAP), alginic acid, salts of alginic acid, polyvinyl acetate phthalate (PVAP) and polyvinyl acetate (PVAc).

12. The pharmaceutical composition according to claim 1, wherein the plasticizers are selected from the group consisting of glycerin, polyethylene glycol, triethyl citrate, tributyl citrate, propyl triacetate and castor oil.

13. The pharmaceutical composition according to claim 1, wherein the other diluents used in the film coating are selected from the group consisting of lactose, starch, mannitol, sodium carboxymethyl cellulose, sodium starch, microcrystalline cellulose and pigments.

14. The pharmaceutical composition according to claim 1, wherein the anti-sticking agents used in the film coating are selected from the group consisting of talc, stearic acid, stearate, sodium stearyl fumarate and glyceryl behenate.
